(19) United States
(12) Patent Application Publication
(10) Pub. No.: US 2026/0260338 A1
(43) Pub. Date: Sep. 3, 2026

(54) SYSTEM AND METHOD FOR DETECTING RETINOPATHY OF PREMATURITY

(71) Applicant: REMIDIO INNOVATIVE SOLUTIONS PRIVATE LIMITED, Bangalore (IN)

(72) Inventors: Divya RAO PARTHASARATHY, Ellicott City, MD (US); Florian MicKael SAVOY, Singapore (SG)

(21) Appl. No.: 18/843,012

(22) PCT Filed: Jan. 4, 2024
(86) PCT No.: PCT/IN2024/050011

§ 371 Date: Aug. 30, 2024

(30) Foreign Application Priority Data
Jan. 4, 2023 (IN) 202311000640

(51) Int. Cl. G06T 7/00; G06T 3/40; G06V 10/46; G06V 10/774; G06V 10/98

(52) U.S. Cl. CPC G06T 7/0012; G06T 3/40; G06V 10/462; G06V 10/774; G06V 10/993; G06T 2207/20081; G06T 2207/20132; G06T 2207/20192; G06T 2207/30041; G06T 2207/30101; G06T 2207/30168; G06V 2201/03

(57) ABSTRACT

Approaches for detecting presence of ROP in an input eye, are described. In an example, an input eye image, corresponding to the input eye, is obtained. Once obtained, the input eye image may undergo a plurality of pre-processing steps including cropping, padding, resizing, and sharpening. Thereafter, the input eye image may be processed based on a view assessment model to select a temporal view image. Then, the input eye image may be processed based on a quality assessment module to ascertain the quality of the input eye image. Once the input eye image is ascertained to be acceptable based on quality standards, the same may be processed based on a categorization model to obtain attribute information to detect the presence of the ROP and performs binary categorization of the input eye image as one of a no referral ROP and a referral ROP.

### BACKGROUND

The development of retinal blood vessels in the eye of an infant is dependent on a plurality of factors. Among other factors, one of the most important factors is the time of birth of the infant. For example, premature birth of infants may hamper or interrupt the development of retinal blood vessels resulting in abnormal development of retinal blood vessels when infant comes out of mother's womb. Due to such abnormal development, a disease named Retinopathy of prematurity (ROP) is caused which is a potentially blinding disease. In lack of early screening of ROP, a severe type of ROP may be caused which eventually results in pulling away of the retina from the wall of the eye and causes blindness. In most cases, ROP screening may be performed under the supervision of highly specialized ophthalmologist using bedside clinical examination equipment. However, such clinical procedures are not suitable for providing accurate and reliable screening facilities to large populations in different geographical locations at minimal cost.

### BRIEF DESCRIPTION OF FIGURES

Systems and/or methods, is accordance with examples of the present subject matter are now described and with reference to the accompanying figures, in which:

FIG. 1A-1B illustrates a training system for training a Retinopathy of Prematurity (ROP) detection model pipeline, as per an example;

FIG. 2 illustrates a Retinopathy of Prematurity (ROP) detection system for detecting presence of ROP in an input eye, as per another example;

FIG. 3 illustrates a method for training the ROP detection model pipeline, as per an example; and

FIGS. 4A-4B illustrates a method for detecting presence of ROP in the input eye image, based on a trained ROP detection model pipeline, as per an example.

### DETAILED DESCRIPTION

Retinopathy of Prematurity (ROP) is a potentially blinding disease that affects prematurely born infants, particularly those who weigh less than 2 kilograms at birth. The human eye contains a lens that focuses images on the inside of the back of the eye, i.e., on the retina. The retina is covered with a network of blood vessels underneath it. These vessels normally grow quickly in the last few weeks before a baby is born. However, due to premature delivery of the baby, the growth of these blood vessels stops and may grow into parts of the eye where they are not intended for. This may lead to the formation of scar tissue which eventually damages the retina and causes a loss of vision, a complication referred to as ROP. To prevent the negative effects of the ROP, early screening of the same is required.

ROP screening is a process that may be performed under the supervision of a specialized ophthalmologist using bedside clinical examination equipment or digital image analysis tools. The screening process involves monitoring changes or patterns in the growth of the retinal blood vessels on the back of the eye or underneath the retina. In addition, due to advancements in machine learning, several machine learning algorithms have been developed to automate the detection of ROP.

However, in case of manual examination, manual examination requires the presence of a specialized pediatric ophthalmologist and expensive equipment enabled with teleophthalmology. The availability of such specialized medical practitioners and equipment is limited, and it may be costly to provide this facility in large numbers of tertiary level healthcare centers, particularly those located in rural areas. On the other hand, automatic examination using machine learning focuses on determining plus disease, which is an advanced stage of ROP. This approach is not sufficient for early detection and screening of ROP.

Therefore, there is a continuous effort in the field of ophthalmology and medical technology to develop systems and methods for efficient, accurate, and cost-effective screening of ROP, particularly in large populations across different geographical locations.

Approaches for detecting presence of ROP in an input eye, are described. The detection of presence of ROP in the input eye is performed using an input eye image which corresponds to a subject which is under evaluation for detecting presence of ROP. In another example, the detection of presence of ROP in the input eye may be performed using a plurality of input eye images which corresponds to different views of the eyes. Examples of such views include, but are not limited to, temporal view, nasal view, disc centered view, macula centered view, inferior view and superior view. The plurality of input eye images may also correspond to images of the input eye of the subject which is under screening. Such input eye images may either be stored in a database repository or may be captured using a camera device.

In one example, the input eye image (or the set of input eye images), corresponding to the input eye, which is to be screened for detecting presence of ROP, is obtained. Once obtained, the input eye image may undergo a plurality of pre-processing steps including cropping, padding, resizing, and sharpening. The purpose of these pre-processing steps is to make the input eye image (or all the input eye images) compatible for assessing the view of eye images. It may be noted that, performing these pre-processing steps are not necessarily essential.

Once the pre-processing steps are performed, the input eye image may be processed based on a view assessment model to determine whether the input eye image have a temporal view. In another example, in case of set of input eye images, the multiple images of eyes may be processed based on the view assessment model to select an input eye image from the set of input eye images having a temporal view. Examples of possible views of the eye images include, but are not limited to, temporal view, macula view, optic disc centered view, inferior view, superior view, and nasal view. In an example, other images having different views rather than the temporal view are discarded. In one example, on identifying none of the images among the set of input eye images belong to temporal view, a prompt message may be displayed to the user using the system. It may be noted that, particularly having temporal view image helps in making the process computationally efficient and accurate.

Continuing with the present example, the input eye image having temporal view may then undergo a plurality of processing steps which includes cropping, padding, resizing, and sharpening. As described above as well, these processing steps are to make the input eye image compatible for further stages of processing, e.g., quality assessment, to detect the presence of ROP. Further, performing these processing steps are not necessarily essential and may be omitted as the case may be.

Once the processing steps are performed, the input eye image may then be processed based on a quality assessment module to ascertain the quality of the input eye image. If the image quality of the input eye image is acceptable, the input eye image may be used for further stages of process to detect the presence of ROP. In an example, if the input eye image is not of acceptable quality, a user may be prompted to capture the input eye image again.

Once the input eye image is ascertained to be acceptable based on quality standards, the same may be processed based on a categorization model to obtain attribute information of the input eye image. In an example, the attribute information corresponds to a plurality of eye image attributes of the input eye image. Examples of such eye image attributes include, but are not limited to, retinal blood vessels location, retinal blood vessel dimension, retinal blood vessel architecture, demarcation line presence, demarcation line location, demarcation line dimension, presence of ridge, location of ridge, dimension of ridge, indicators indicating partial retinal detachment, indicators indicating total retinal detachment and many more.

Subsequently, based on the obtained attribute information, the categorization model detects the presence of the ROP and performs binary categorization of the input eye image as one of a no referral ROP and a referral ROP. It may be noted that, although limited examples of eye image attributes indicating presence or absence of ROP are described above, other such examples would still be withing the scope of the present subject matter. In one example, the attribute information may be used as a measurement parameter for ascertaining presence of ROP, as described subsequently.

In addition to the result of detection of presence of ROP in the input eye image, a visualization output may also be generated. In one example, the visualization output may be in the form of an activation map. The activation map thus obtained may indicate or highlight areas of abnormality in the input eye image which represents those areas or salient regions which lead to designation of input eye as referred ROP eye. These and other aspects have been discussed in further detail later in the present description.

It may be noted that the above-mentioned determinations involving view assessment, quality assessment, obtaining the attribute information, detecting presence of ROP may involve a variety of models such as the view assessment model, quality assessment model, and the categorization model. In one example, each of the aforementioned models are machine learning based models. In an example, the machine learning model may be a deep learning model. Although having been described as unique or separate models, the view assessment model, the quality assessment model, and the categorization model may be implemented as a ROP detection model pipeline for the detection of ROP in the subject eye. It may also be noted that a ROP detection system comprising the plurality of machine learning algorithm (such as quality assessment model, view assessment model and categorization model) further includes an analysis engine which performs one or more intermediate functions, such as pre-processing of input eye image and processing of input eye image, without deviating from the scope of the present subject matter.

The machine learning models within the ROP detection model pipeline may be trained based on a variety of training dataset. For example, the view assessment model may be trained based on training images having different views, e.g., images have temporal view, macula view, optic disc centered view, inferior view, superior view, and nasal view. Similarly, the quality assessment model may be trained based on a variety of training images having variety of resolution, contrast, clarity, or other such attributes. In a similar manner, the categorization model may be trained based on training images which are associated with ROP and the training images which are free of ROP, or not associated with ROP.

The categorization model may also be trained based on training attribute information that may be obtained through clinical history, comprehensive eye examination and investigational modalities that include but not limited to optical coherence tomography, visual fields, intraocular pressure measurements, pachymetry etc. In an example, the categorization model includes two sub-models, i.e., a binary classification model which is trained to detect presence or absence or ROP and a categorical classification model which is trained to categorize eye image in various stages which may include but are not limited to Stage 1, Stage 2, Stage 3, Stage 4, Stage 5, A-ROP (Aggressive posterior ROP) and Smouldering ROP. In an example, the categorical classification model may be used only during training to supplement in the accuracy of detection of ROP by the binary classification model. Although the training has been described in the context of the view assessment model, quality assessment model, and the categorization model, such similar training procedures may be performed for other models that may be implemented within the ROP detection model pipeline. Such processes would still fall within the scope of the present subject matter without limitation.

The present approaches overcome the above-mentioned technical advantages. For example, the above-mentioned approaches may be implemented in a single device for effective ROP screening. Since no specialized equipment or skill is required, a system implementing the present approaches is mobile, cost-effective, and accurate for the purposes of ROP detection. For example, an implementing system allows for screening without expert knowledge and is performable on portable retinal camera itself, while ensuring a desired and functional level of accuracy.

The explanation provided above and the examples that are discussed further in the current description are exemplary only. For instance, some of the examples may have been described in which only one image is considered, either in training or in inference stage. However, the current approaches may be adopted for other instances or situations as well, such as a set of input eye images, a set of training eye images may be used, or such without deviating from the scope of the present subject matter.

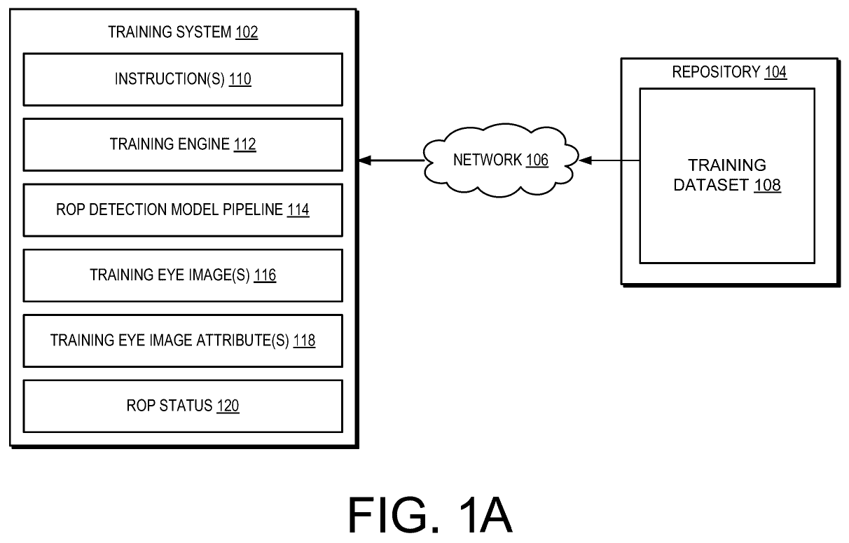

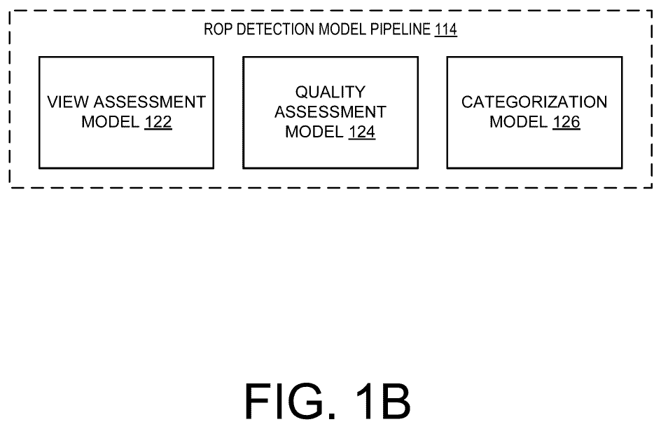

The manner in which models implemented within the ROP detection model pipeline are trained and used for identifying presence of ROP in the input eye is explained in detail with respect to FIGS. 1A-4B. While aspects of described systems may be implemented in any number of different electronic devices, environments, and/or implementation, the examples are described in the context of the following example device(s). In another example, the aspects of the present subject matter may also be implemented by a standalone device having executable instructions. It may be noted that drawings of the present subject matter shown here are for illustrative purposes and are not to be construed as limiting the scope of the subject matter claimed.

FIG. 1A illustrates a training system 102 comprising a processor or memory (not shown), for training models within the ROP detection model pipeline. In an example, the training system 102 (referred to as system 102) may be communicatively coupled to a repository 104 through a network 106. The repository 104 may further include training dataset 108. The training dataset 108 may include a plurality of training images that may be used for training the ROP detection model pipeline. In an example, these pluralities of training images are those images which are captured previously while manual screening of the subject with corresponding ROP category annotated.

In another example, along with plurality of training images, the training dataset 108 may further include training attribute information and corresponding ROP category for each of the plurality of training images. The training attribute information corresponds to a plurality of training eye image attributes. In an example, the training eye image attributes may include retinal blood vessels location, retinal blood vessel dimension, retinal blood vessel architecture, demarcation line presence, demarcation line location, demarcation line dimension, presence of ridge, location of ridge, dimension of ridge, indicators indicating partial retinal detachment, indicators indicating total retinal detachment and many more.

Although depicted as being obtained from a single repository, such as repository 104, the training dataset 108 may also be obtained from multiple other sources without deviating from the scope of the present subject matter. In such cases, each of such multiple repositories may be interconnected through a network, such as network 106.

The network 106 may be a private network or a public network and may be implemented as a wired network, a wireless network, or a combination of a wired and wireless network. The network 106 may also include a collection of individual networks, interconnected with each other and functioning as a single large network, such as the Internet. Examples of such individual networks include, but are not limited to, Global System for Mobile Communication (GSM) network, Universal Mobile Telecommunications System (UMTS) network, Personal Communications Service (PCS) network, Time Division Multiple Access (TDMA) network, Code Division Multiple Access (CDMA) network, Next Generation Network (NGN), Public Switched Telephone Network (PSTN), Long Term Evolution (LTE), and Integrated Services Digital Network (ISDN).

The system 102 may further include instructions 110 and a training engine 112. In an example, the instructions 110 are fetched from a memory and executed by a processor included within the system 102. The training engine 112 may be implemented as a combination of hardware and programming, for example, programmable instructions to implement a variety of functionalities. In examples described herein, such combinations of hardware and programming may be implemented in several different ways. For example, the programming for the training engine 112 may be executable instructions, such as instructions 110. Such instructions may be stored on a non-transitory machine-readable storage medium which may be coupled either directly with the system 102 or indirectly (for example, through networked means). In an example, the training engine 112 may include a processing resource, for example, either a single processor or a combination of multiple processors, to execute such instructions. In the present examples, the non-transitory machine-readable storage medium may store instructions, such as instructions 110, that when executed by the processing resource, implement training engine 112. In other examples, the training engine 112 may be implemented as electronic circuitry.

The instructions 110, when executed by the processing resource, cause the training engine 112 to train the ROP detection model pipeline 114 based on the training dataset 108. The system 102 may further include a training eye image(s) 116, a training eye image attribute(s) 118, a ROP category 120. In an example, the system 102 may obtain training dataset 108 corresponding to a single training eye image from the repository 104, and the information pertaining to that is stored as training eye image(s) 116, training eye image attribute(s) 118, and ROP category 120 in the system 102.

As described previously, the ROP detection model pipeline 114 (referred to as model pipeline 114) may further include a plurality of machine learning models. An example of such machine learning models include deep learning models. For the sake of explanation, the current approaches for detection of presence of ROP has been described with the different steps being performed using one or more deep learning models, as examples. Although the present examples have been described in relation to deep learning models, the aforementioned approaches may also be implemented using other machine-learning models. It may also be noted that any explanation provided in conjunction with deep learning models is applicable to other machine learning models, without limitations and without deviating from the scope of the present subject matter. Such examples have not been described for sake of brevity. The manner in which the training of the plurality of the models within the model pipeline 114 may be performed is further described in conjunction with FIG. 1B.

FIG. 1B depicts example deep learning models that may be implemented within the model pipeline 114. In one example, the model pipeline 114 may include a view assessment model 122, a quality assessment model 124, and a categorization model 126. It may be noted that the model pipeline 114 may include other deep learning models (such as pre-processing model and processing model which are not shown in FIG. 1B) as well for implementing various other functions. It may also be the case that one or more models may be implemented so as to perform a combination of one or more functions. Such variations and combinations would still be examples of the present subject matter without limitations.

With respect to training the view assessment model 122, the training eye image(s) 116 may be used wherein the training eye image(s) 116 may include images having different views, e.g., temporal view, nasal view, disc centered view, macula centered view, inferior view, and superior view. For training the quality assessment model 124, the training eye image(s) 116 may include images having higher resolution, contrast, clarity, or other such attributes. The quality assessment model 124 is trained to assess the quality of the input eye images so that the images having low quality may be discarded and only good quality images having higher quality are considered for further processing.

The categorization model 126 in turn may be trained based on training eye image(s) 116 which identify the attribute information corresponding to the plurality of eye image attributes within the training eye image(s) 116. The categorization model 126 may also be trained on training eye images which are associated with ROP and images which are not associated with ROP as part of the training eye image(s) 116. There is a category indicator, such as ROP category 120, which is associated with each of the training eye image(s) 116 representing the state of corresponding training eye image.

In an example, the categorization model includes two sub-models, i.e., a binary classification model which is trained to detect presence or absence or ROP and a categorical classification model which is trained to categorize eye image in various stages, e.g., Stage 1, Stage 2, Stage 3, Stage 4, Stage 5, A-ROP (Aggressive posterior ROP) and Smouldering ROP. In an example, the categorical classification model may be used only during training to supplement in the accuracy of detection of ROP by the binary classification model.

As will be discussed subsequently, the view assessment model 122, the quality assessment model 124, and the categorization model 126 when trained may be used to perform a variety of task either sequentially or concurrently based on which presence of ROP within a subject eye may be ascertained.

As described above as well, the training of the view assessment model 122, the quality assessment model 124, and the categorization model may be performed in any order and may be performed at different instants. As may be understood, although one or more common training datasets may be used, the training of any one of the deep learning models in the model pipeline 114 is independent from the training of another model.

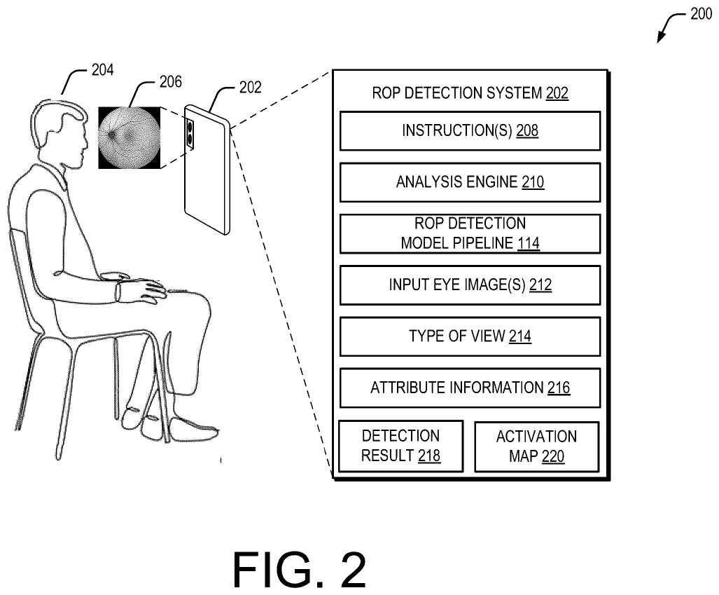

In an example, once trained, the model pipeline 114 may be utilized for categorizing an input eye image as one of a no-referral ROP and a referral ROP. The manner in which the model pipeline 114 may be used for detection of ROP within the subject eye is further described in conjunction with FIG. 2.

FIG. 2 illustrates an environment 200 with a Retinopathy of Prematurity (ROP) detection system 202 for determining a ROP category of an input eye image 204 of a subject 206. In an example, the ROP detection system 202 (referred to as system 202) includes a mobile phone, tablet, or any other portable computing device. In an example, the portable computing device attached onto the system 202 is capable of capturing retinal images of the subject's eye. The input eye image 204 may be an image of an eye of the subject 206 who is under screening for the diagnosis of ROP. In an example, the input eye image 204 is a retinal image. In an example, the system 202 may analyze a plurality of eye image attributes of the input eye image 204 based on the trained model pipeline 114.

Similar to the system 102, the system 202 may further include instructions 208 and an analysis engine 210. In an example, the instructions 208 are fetched from a memory and executed by a processor included within the system 202. The analysis engine 210 may be implemented as a combination of hardware and programming, for example, programmable instructions to implement a variety of functionalities. In examples described herein, such combinations of hardware and programming may be implemented in several different ways. For example, the programming for the analysis engine 210 may be executable instructions, such as instructions 208. Such instructions 208 may be stored on a non-transitory machine-readable storage medium which may be coupled either directly with the system 202 or indirectly (for example, through networked means). In an example, the analysis engine 210 may include a processing resource, for example, either a single processor or a combination of multiple processors, to execute such instructions. In the present examples, the non-transitory machine-readable storage medium may store instructions, such as instructions 208, that when executed by the processing resource, implement analysis engine 210. In other examples, the analysis engine 210 may be implemented as electronic circuitry.

In one example, the analysis engine 210 may utilize the trained model pipeline 114 to ascertain whether ROP is present within the subject eye based on the processing of the input eye image 204 of the subject 206. It may be noted that the model pipeline 114 may be trained by way of the approach discussed in conjunction with FIGS. 1A-1B. As also described previously, the model pipeline 114 may further include trained view assessment model 122, quality assessment model 124, and the categorization model 126.

The system 202 may further include an input eye image(s) 212, type of view 214, attribute information 216, detection result 218 and activation map 220. It may be noted that the aforesaid data elements are generated by the analysis engine 210 using the model pipeline 114 and in response to the execution of the instruction(s) 208. These aspects and further details are discussed in the following paragraphs.

In operation, an input eye image, such as the input eye image 204 of an eye of the subject 206 who is under screening for the detection of presence of ROP, may be obtained. For example, the input eye image 204 may be captured through any image sensing sub-system that may be present within the system 202. In an example, the image sensing sub-system may be a retinal camera device which is either installed on the system 202 itself or may be removably integrated with the system 202. In another example, instead of having a single input eye image, a set of input eye images may be obtained. Each of the images of the set of input eye images may correspond to various views possible for an eye image. The set of input eye images also corresponds to the subject eye who is under screening for the detection of presence of ROP.

Once the input eye image 204 is obtained, the analysis engine 210 may perform a plurality of pre-processing steps on the input eye image 204. Examples of such pre-processing steps include, but are not limited to, cropping, padding, resizing, and sharpening. The objective of these pre-processing steps is to make the input eye image 204 compatible for further processing stages and to remove unnecessary portions of the input eye image 204. For example, cropping is to remove or adjust the outside borders or edges of the input eye image 204 to improve framing or composition. Specifically, via cropping the unnecessary parts of the input eye image 204 are removed. Similarly, other pre-processing steps are performed to improve the compatibility of the input eye image 204 for the further stages, e.g., view assessment, of processing.

Continuing further, the input eye image 204 is further processed to assess the view of the input eye image 204. In one example, analysis engine 210 may utilize the trained view assessment model 122 of the model pipeline 114 for ascertaining a type of view, such as type of view 214, of the input eye image 204. In an example, the trained view assessment model 122 assesses various features of the input eye image 204 to determine the view of the input eye image. Examples of various views possible for the input eye image 204 include, but are not limited to, temporal view, nasal view, disc centered view, macula centered view, inferior view, and superior view. Further, in one example, in case of set of input eye images, the type of view of each of the input eye images may be ascertained.

Once the type of view 214 of the input eye image 204 is ascertained, if the ascertained type of view 214 is temporal view, the input eye image 204 may be processed by the analysis engine 210 using the ROP detection model pipeline. In an example, if the input eye image is not of temporal view, the user or the subject 206 may be prompted by displaying an indicator on a display of the system 202 to capture another input eye image 204 or may choose to proceed with the initially captured or obtained input eye image 204. In case of set of input eye images obtained, among images included in the set of input eye images, an image having temporal view is selected and is designated as the input eye image 204.

Continuing further, input eye image 204 having temporal view is subjected to a plurality of processing steps. For example, the analysis engine 210 performs the plurality of processing steps on the input eye image 204 to make it compatible for further stages of processing. As described above as well, the plurality of processing steps include, but are not limited to, cropping, padding, resizing, and sharpening.

Once the input eye image 204 is processed, the input eye image 204 is processed to assess quality of the input eye image 204 using the trained model pipeline 114. In one example, analysis engine 210 may utilize the trained quality model 124 of the model pipeline 114 for ascertaining a quality score for the input eye image 204. In an example, the quality score depicts the level of acceptance of the input eye image. For example, images having higher quality score are accepted and images having lower quality score are discarded. In an example, high quality images are preferred as these images include feature details clearer.

Returning to the present example, once the quality score of the input eye image 204 is determined, if the determined quality score is greater than a threshold score, the input eye image 204 may be processed by the analysis engine 210 using the categorization model to detect the presence of ROP in the subject eye. In an example, if the determined quality score is less than the threshold score, the user or the subject 206 may be prompted by displaying an indicator on the display of the system 202 to capture another input eye image 204 or may choose to proceed with the initially captured or obtained input eye image 204. Both such examples are complimentary and as such have no impact on the scope of the present subject matter. It may be understood that ascertaining the quality of the input eye image 204 may rely on various features or attributes of the input eye image 204, as detected by the quality assessment model 124. It may be noted that, in an example, the user 206 may elect to proceed with subsequent process based on the input eye image 204 without assessing its quality, without deviating from the scope of the present subject matter.

The input eye image 204 (once determined as acceptable as the case may be), may be further processed by the analysis engine 210 using the trained model pipeline 114 to identify attribute information, such as attribute information 216 of the input eye image 204. In one example, the analysis engine 210 may utilize the trained categorization model 126 of the model pipeline 114 to identify the attribute information 216 of the input eye image 204. In an example, the attribute information 216 corresponds to a plurality of eye image attributes which individually or combinedly indicate either presence or absence of ROP in the subject eye.

To this end, the analysis engine 210 may, using the categorization model 126, identify one or more eye image attributes. Examples of the eye image attributes include, but are not limited to, retinal blood vessels location, retinal blood vessel dimension, retinal blood vessel architecture, demarcation line presence, demarcation line location, demarcation line dimension, presence of ridge, location of ridge, dimension of ridge, indicators indicating partial retinal detachment, indicators indicating total retinal detachment and many more.

Based on the attribute information 216 thus determined using the trained categorization model 126, the analysis engine 210 may further process the attribute information 216 based on the categorization model 126 to determine a detection result 218 for the input eye image 204 corresponding to the patient's eye. In an example, the detection result represents absence or presence of ROP within the input eye image 204 of the subject eye. In another example, in case of multiple input eye images, the analysis engine 210 may determine the detection result 218 representing absence or presence of ROP in the patient's eye as a whole by considering all the input eye images. Based on the detection result 218, the analysis engine 210 categorize the input eye image 204 as one of the referral ROP category and the Non-referral ROP category.

It may be noted that the detection result 218 thus determined may be used to provide a further referral for treatment, or other intervention, as may be required. For example, the detection result 218 may be indicative of a diagnosis of ROP. The detection result 218 may indicate one of the following states: referral ROP or non-referral ROP. Based on the state represented by the detection result 218, appropriate action may be taken. Although explained as being obtained by processing above-described examples of eye image attributes included in the attribute information 216, the detection of presence of ROP may be performed by considering any other eye image attributes without deviating from the scope of the present subject matter. Such examples would still fall within the scope of the present subject matter, without any limitation.

Once all the results of processing based on the ROI portions are obtained, the identified resultant category for the input eye image 204 then may be displayed on the display device of the system 202 to indicate the ROP category of the subject under screening so that further steps of treatment are practiced for curing the disease. In an example, the detection result 218 being displayed on a per eye, per subject basis, or as a combination thereof. In furtherance to this, the analysis engine 210 may also generate the activation map 220 to displayed on the display device of the system 202. In an example, the activation map 220 depicts salient regions within the input eye image 204 which triggered the detection of ROP within the input eye image 204 of the subject 206. Further, the displayed activation map may also be further used by medical practitioner to identify the regions which have caused the disease.

In another example, the system 202 may be communicatively coupled to a central computing server through a network (not shown in FIG. 2). The network may be a private network or a public network and may be implemented as a wired network, a wireless network, or a combination of a wired and wireless network, and may be similar to the network 106 (as depicted in FIG. 1A). All the above disclosed steps which may be performed by the analysis engine 210 of the system 202, may be implemented or performed by the central computing server on behalf of the system 202 to reduce computing load on edge of the network.

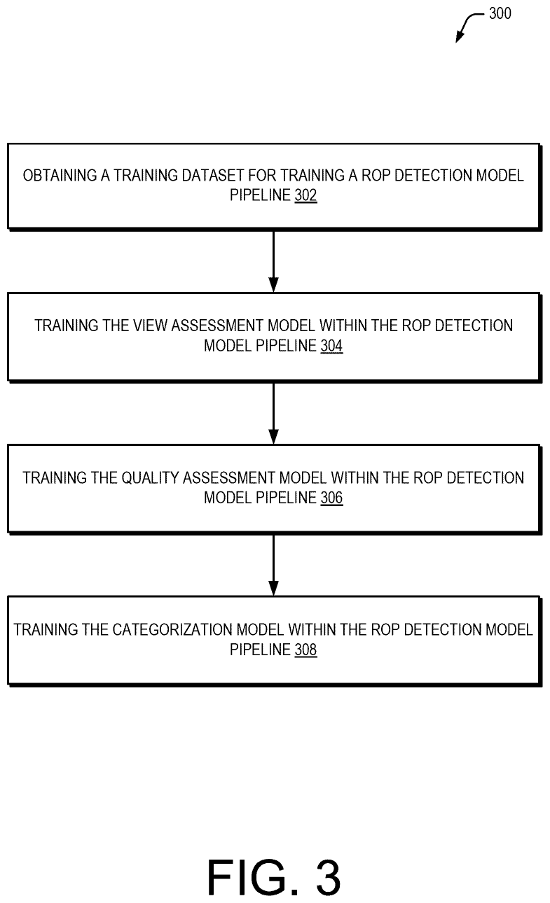

FIG. 3 illustrates an example method 300 for training a ROP detection model pipeline, in accordance with examples of the present subject matter. The order in which the above-mentioned method is described is not intended to be construed as a limitation, and some of the described method blocks may be combined in a different order or may be even performed concurrently to implement the method, or alternative method.

Furthermore, the above-mentioned method may be implemented in a suitable hardware, computer-readable instructions, or combination thereof. The steps of such method may be performed by either a system under the instruction of machine executable instructions stored on a non-transitory computer readable medium or by dedicated hardware circuits, microcontrollers, or logic circuits. For example, the method may be performed by a training system, such as system 102. In an implementation, the method may be performed under an “as a service” delivery model, where the system 102, operated by a provider, receives programmable code. Herein, some examples are also intended to cover non-transitory computer readable medium, for example, digital data storage media, which are computer readable and encode computer-executable instructions, where said instructions perform some or all the steps of the above-mentioned method.

In an example, the method 300 may be implemented by the system 102 for training several deep learning models based on a training dataset, such as training dataset 108. At block 302, training dataset for training a ROP detection model pipeline may be obtained. For example, the system 102 may obtain training dataset 108. The training dataset 108 may be obtained through a repository, such as the repository 104. In one example, the training dataset 108 may include a training eye image(s) (stored as training eye image(s) 116 in system 102), a training eye image attribute(s) (stored as training eye image attribute(s) 118 in system 102), and the ROP category 120 based on which different models in the model pipeline 114 are to be trained. In an example, the model pipeline 114 may include view assessment model 122, quality assessment model 124, and categorization model 126.

At block 304, the view assessment model which is present within the ROP detection model pipeline may be trained. In one example, the training engine 112 may train the view assessment model 122 using the training eye image(s) 116 wherein the training eye image(s) 116 may include images having different views, e.g., temporal view, nasal view, disc centered view, macula centered view, inferior view, and superior view. The view assessment model 122 is thus trained to ascertain the view of the input eye image.

At block 306, the quality assessment model of the ROP detection model pipeline may be trained. For example, the training engine 112 of the system 102 may train the quality assessment model 124 based on the training eye image(s) 116 which may include images having higher resolution, contrast, clarity, or other such attributes. The quality assessment model 124 is trained to assess the quality of the input eye images so that the images having low quality may be discarded and only good quality images having higher quality are considered for further processing.

At block 308, the categorization model of the ROP detection model pipeline may be trained. For example, the training engine 112 of the system 102 may train the categorization model 126 based on training eye image(s) 116 which identify the attribute information corresponding to the plurality of eye image attributes within the training eye image(s) 116. The categorization model 126 may also be trained on training eye images which are associated with ROP and images which are not associated with ROP as part of the training eye image(s) 116. There is a category indicator, such as ROP category 120, which is associated with each of the training eye image(s) 116 representing the state of corresponding training eye image.

In an example, the categorization model includes two sub-models, i.e., a binary classification model which is trained to detect presence or absence or ROP and a categorical classification model which is trained to categorize eye image in various stages, e.g., healthy eye, early ROP, intermediate ROP, and advanced ROP. In an example, these sub-models are either trained sequentially or concurrently as per the requirement. In an example, the categorical classification model may be used only during training to supplement in the accuracy of detection of ROP by the binary classification model.

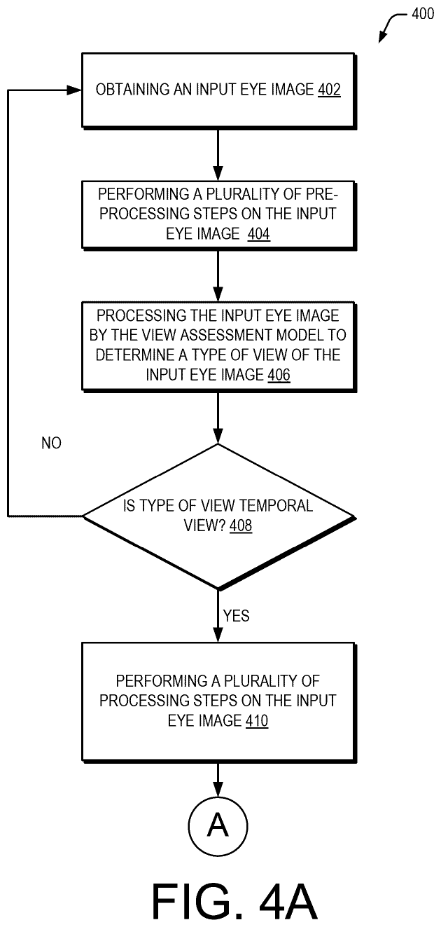

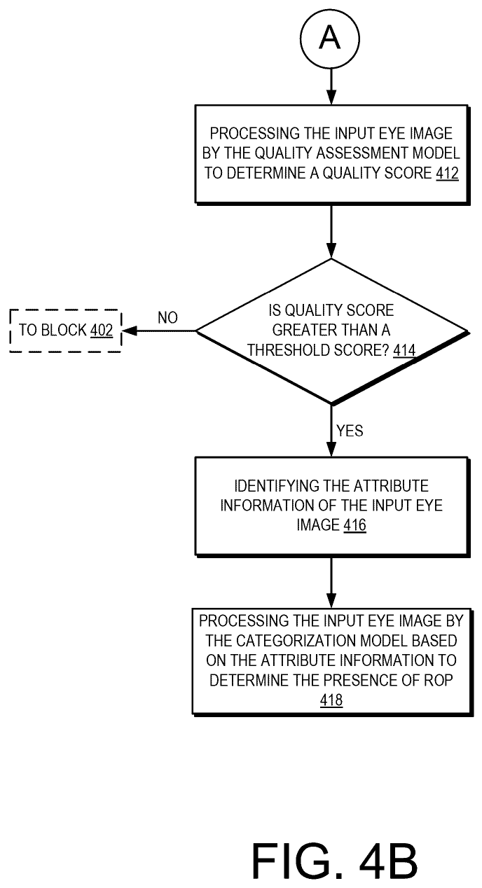

In an example, once trained, the model pipeline 114 may be utilized for categorizing an input eye image as one of a no-referral ROP and a referral ROP. The method steps involved in categorizing the input eye image as one of referral ROP and non-referral ROP are further described in conjunction with FIG. 4A-4B.

FIGS. 4A-4B illustrates example method 400 for categorizing an input image under one of referral ROP category and non-referral ROP category. Similar to FIG. 3, the order in which the above-mentioned method is described is not intended to be construed as a limitation, and some of the described method blocks may be combined in a different order to implement the method, or alternative method. Based on the present approaches as described in the context of the example method 400, the eye image attributes of an input eye image is analyzed based on the trained model pipeline 114.

Further, the above-mentioned method 400 may be implemented in a suitable hardware, computer-readable instructions, or combination thereof. The steps of such method may be performed by either a system under the instruction of machine executable instructions stored on a non-transitory computer readable medium or by dedicated hardware circuits, microcontrollers, or logic circuits. For example, the method may be performed by a ROP detection system, such as system 202. In an implementation, the method may be performed under an “as a service” delivery model, where the system 202, operated by a provider, receives programmable code. Herein, some examples are also intended to cover non-transitory computer readable medium, for example, digital data storage media, which are computer readable and encode computer-executable instructions, where said instructions perform some or all the steps of the above-mentioned method.

At block 402, an input eye image is obtained. For example, the input eye image 204 may be captured through any image sensing sub-system that may be present within the system 202. In an example, the image sensing sub-system may be a retinal camera device which is either installed on the system 202 itself or may be removably integrated with the system 202. In another example, instead of having a single input eye image, a set of input eye images may be obtained. Each of the images of the set of input eye images may correspond to various views possible for an eye image. The set of input eye images also corresponds to the subject eye who is under screening for the detection of presence of ROP. In another example, the input eye image 204 may be obtained from a database repository (not shown in FIG. 2) storing samples of eye images to be tested for detecting presence of ROP.

At block 404, a plurality of pre-processing steps may be performed on the input eye image thus obtained. For example, the analysis engine 210 may perform a plurality of pre-processing steps on the input eye image 204. Examples of such pre-processing steps include, but are not limited to, cropping, padding, resizing, and sharpening. The objective of these pre-processing steps is to make the input eye image 204 compatible for further processing stages and to remove unnecessary portions of the input eye image 204. For example, cropping is to remove or adjust the outside edges of the input eye image 204 to improve framing or composition. Specifically, via cropping the unnecessary parts of the input eye image 204 are removed. Similarly, other pre-processing steps are performed to improve the compatibility of the input eye image 204 for the further stages, e.g., view assessment, of processing.

At block 406, a type of view of the input eye image is determined by processing the input eye image. For example, analysis engine 210 may utilize the trained view assessment model 122 of the model pipeline 114 for ascertaining a type of view, such as type of view 214, of the input eye image 204. In an example, the trained view assessment model 122 assesses various features of the input eye image 204 to determine the view of the input eye image. Examples of various views possible for the input eye image 204 include, but are not limited to, temporal view, nasal view, disc centered view, macula centered view, inferior view, and superior view.

At block 408, a determination may be made to ascertain whether the type of view of the input eye image is temporal or not. For example, if the ascertained type of view 214 is temporal view, the input eye image 204 may be processed by the analysis engine 210 using the ROP detection model pipeline (‘Yes’ path from block 408), as will be described in later steps. If, however, the input eye image 204 is not of temporal view, the user or the subject 206 may be prompted by displaying an indicator on a display of the system 202 to capture or obtain another input eye image 204 (‘No’ path from block 408) or may choose to proceed with the initially captured or obtained input eye image 204. In case of set of input eye images obtained, among images included in the set of input eye images, an image having temporal view is selected and is designated as the input eye image 204.

At block 410, a plurality of processing steps may be performed on the input eye image. For example, the analysis engine 210 performs the plurality of processing steps on the input eye image 204 to make it compatible for further stages of processing. As described above as well, the plurality of processing steps include, but are not limited to, cropping, padding, resizing, and sharpening.

At block 412, the input eye image may be further processed based on the quality assessment model to determine its acceptability. For example, analysis engine 210 may utilize the trained quality assessment model 124 of the model pipeline 114 for ascertaining a quality score for the input eye image 204. In an example, the quality score depicts the level of acceptance of the input eye image. For example, images having higher quality score are accepted and images having lower quality score are discarded. In an example, high quality images are preferred as these images include feature details clearer.

At block 414, a determination is made whether the determined quality score of the input eye image is acceptable or not. For example, if the determined quality score is greater than the threshold score, the input eye image 204 may be processed by the analysis engine 210 using the categorization model to detect the presence of ROP in the subject eye (‘Yes’ path from block 414). If, however, the determined quality score is less than the threshold score, the user or the subject 206 may be prompted by displaying an indicator on the display of the system 202 to capture or obtain another input eye image 204 (‘No’ path from block 414) or may choose to proceed with the initially captured or obtained input eye image 204. Both such examples are complimentary and as such have no impact on the scope of the present subject matter. It may be understood that ascertaining the quality of the input eye image 204 may rely on various features or attributes of the input eye image 204, as detected by the quality assessment model 124. It may be noted that, in an example, the user 206 may elect to proceed with subsequent process based on the input eye image 204 without assessing its quality, without deviating from the scope of the present subject matter.

At block 416, the input eye image may be further processed based on the categorization model to identify the attribute information of the input eye image. For example, the analysis engine 210 may utilize the trained categorization model 126 of the model pipeline 114 to identify the attribute information 216 of the input eye image 204. In an example, the attribute information 216 corresponds to a plurality of eye image attributes which individually or combinedly indicate either presence or absence of ROP in the subject eye.

To this end, the analysis engine 210 may, using the categorization model 126, identify one or more eye image attributes. Examples of the eye image attributes include, but are not limited to, retinal blood vessels location, retinal blood vessel dimension, retinal blood vessel architecture, demarcation line presence, demarcation line location, demarcation line dimension, presence of ridge, location of ridge, dimension of ridge, indicators indicating partial retinal detachment, indicators indicating total retinal detachment and many more.

At block 418, the determined attribute information may be further processed based on the categorization model to determine a detection result indicating the presence or absence of ROP in the subject eye. For example, the analysis engine 210 may further process the attribute information 216 based on the categorization model 126 to determine a detection result 218 for the input eye image 204. In an example, the detection result represents absence or presence of ROP within the input eye image 204 of the subject eye. Based on the detection result 218, the analysis engine 210 categorize the input eye image 204 as one of the referral ROP category and the Non-referral ROP category.

It may be noted that the detection result 218 thus determined may be used to provide a further referral for treatment, or other intervention, as may be required. For example, the detection result 218 may be indicative of a diagnosis of ROP. The detection result 218 may indicate one of the following states: referral ROP or non-referral ROP. Based on the state represented by the detection result 218, appropriate action may be taken. Although explained as being obtained by processing above described examples of eye image attributes included in the attribute information 216, the detection of presence of ROP may be performed by considering any other eye image attributes without deviating from the scope of the present subject matter. Such examples would still fall within the scope of the present subject matter, without any limitation.

Once all the results of processing based on the ROI portions are obtained, the identified resultant category for the input eye image 204 then may be displayed on the display device of the system 202 to indicate the ROP category of the subject 206 under screening so that further steps of treatment are practiced for curing the disease. In an example, the detection result 218 being displayed on a per eye, per subject basis, or as a combination thereof.

In furtherance to this, the analysis engine 210 may also generate the activation map 220 to displayed on the display device of the system 202. In an example, the activation map 220 depicts salient regions within the input eye image 204 which triggered the detection of ROP within the input eye image 204 of the subject 206. Further, the displayed activation map may also be further used by medical practitioner to identify the regions and extent of the disease.

Although examples for the present disclosure have been described in language specific to structural features and/or methods, it is to be understood that the appended claims are not necessarily limited to the specific features or methods described. Rather, the specific features and methods are disclosed and explained as examples of the present disclosure.

1. A system comprising:
a processor; and
an analysis engine coupled to the processor, wherein the analysis engine is for:
obtaining an input eye image, wherein the input eye image corresponds to a subject eye which is under evaluation for detecting presence of Retinopathy of Prematurity (ROP);
using a ROP detection model pipeline, wherein the ROP detection model pipeline is trained based on a training dataset comprising training images associated with the ROP, and training attribute information which corresponds to a plurality of training eye image attribute, wherein the ROP detection model pipeline is for:
determining a type of view of the input eye image;
performing a plurality of processing steps on the input eye image to make it compatible for further processing upon determining the type of view as a temporal view,
identifying an attribute information from the input eye image, wherein the attribute information corresponds to a plurality of input eye image attributes; and
determining a detection result indicating presence of ROP within the subject eye based on the attribute information.

2. The system as claimed in claim 1, wherein the analysis engine is to:
performing a plurality of pre-processing step on the input eye image to make the input eye image compatible for the view assessment, wherein the plurality of pre-processing steps comprises cropping, padding, resizing, and sharpening the edges of the input eye image.

3. The system as claimed in claim 1, wherein the plurality of processing steps comprises cropping, padding, resizing, and sharpening the edges of the input eye image.

4. The system as claimed in claim 1, wherein the analysis engine is to use the ROP detection model pipeline for:
discarding the input eye image when the type of view of the input eye image is other than temporal view.

5. The system as claimed in claim 1, wherein the analysis engine is for:
obtaining a set of input eye images, wherein each of the images of the set of input eye images corresponds to different views of the user's eye, wherein the views comprises a temporal view, nasal view, disc centered view, macula centered view, inferior view, and superior view;
using the ROP detection model pipeline for;
determine determining the view of each of the images of the set of input eye images;
selecting an image from the set of input eye images having temporal view to be designated as input eye image based on the determined view.

6. The system as claimed in claim 1, wherein the ROP detection model pipeline comprises a plurality of deep learning models selected from a group comprising a view assessment model, a quality assessment model, and a categorization model.

7. The system as claimed in claim 1, wherein the analysis engine is to use the ROP detection model pipeline for:
assessing quality of the input eye image to generate a quality score;
extracting the attribute information from the input eye image upon determining quality score to be greater than the threshold quality score; or
discarding the input eye image upon determining the quality score to be less than a threshold quality score; and
prompting a user to obtain or capture new input eye image.

8. The system as claimed in claim 1, wherein the analysis engine is for further:
generating an activation map depicting salient regions within the input eye image which triggered the detection of ROP within the input eye image of the subject eye.

9. The system as claimed in claim 1, wherein the plurality of input eye image attributes comprises retinal blood vessel location, retinal blood vessel dimension, retinal blood vessel architecture, demarcation line presence, demarcation line location, demarcation line dimension, presence of ridge, location of ridge, dimension of ridge, indicators indicating partial retinal detachment, and indicators indicating total retinal detachment and many more.

10. A method comprising:
obtaining a training information comprising a training eye image and training attribute information corresponding to plurality of eye image characteristics, wherein the training eye image is associated with Retinopathy of prematurity (ROP); and
training a ROP detection model pipeline based on the training information comprising training eye image, training attribute information, and a ROP category of the training eye image, wherein the training attribute information corresponds to a plurality of training eye image attributes.

11. The method as claimed in claim 10, wherein the plurality of training eye image attributes comprises retinal blood vessel location, retinal blood vessel dimension, retinal blood vessel architecture, demarcation line presence, demarcation line location, demarcation line dimension, presence of ridge, location of ridge, dimension of ridge, indicators indicating partial retinal detachment, indicators indicating total retinal detachment and many more.

12. The method as claimed in claim 10, wherein the ROP detection model pipeline when trained based on the training eye image and corresponding training attribute information is to determine a detection result indicating presence of ROP within the within an input eye image of a subject eye which is under evaluation.

13. The method as claimed in claim 10, wherein the ROP detection model pipeline comprises a plurality of deep learning models selected from a group comprising a view assessment model, a quality assessment model, and a categorization model.

14. The method as claimed in claim 10, wherein the ROP detection model pipeline when trained is to assess quality of the input eye image to discard low quality images.

15. The method as claimed in claim 10, wherein the ROP detection model pipeline when trained is to determine type of view of the input eye image to accept only temporal view image, wherein the input eye image have one of a temporal view, nasal view, disc centered view, macula centered view, inferior view, and superior view.
